(19) United States
(12) Patent Application Publication
(10) Pub. No.: US 2015/0105279 A1
(43) Pub. Date: Apr. 16, 2015

(54) PRODUCT SELECTION USING GENETIC ANALYSIS

(71) Applicant: Gene Onyx Limited, Road Town, Tortola (VG)

(72) Inventors: Christofer Touumazou, London (GB); Belinda Nedjai, London (GB); Calvin Sim, Kowloon (HK)

(21) Appl. No.: 14/367,256

(22) PCT Filed: Nov. 1, 2012
(86) PCT No.: PCT/GB2012/052725

§ 371 Date: Jun. 20, 2014

(30) Foreign Application Priority Data
Dec. 20, 2011 (GB) 1121917.7
Dec. 22, 2011 (GB) 1122123.1

(51) Int. Cl. C12Q 1/68; G06F 19/28

(52) U.S. Cl. CPC C12Q 1/6883; G06F 19/28; C12Q 2600/148; C12Q 2600/156
USPC 506/9; 435/6.11; 702/19

(57) ABSTRACT

A method of assessing the suitability of a set of cosmetic and/or nutricosmetic and/or skin care products for an individual. The method comprises testing a sample of genetic material for an individual to identify the presence or absence of single-nucleotide polymorphisms at a predefined set of single-nucleotide locations. One or more weights for each location are identified in dependence upon the presence or absence of a single-nucleotide polymorphism at the location and the single-nucleotide location weights used in order to determine a product score for each of said products, a score being indicative of the suitability of a product to the individual.

### TECHNICAL FIELD

The present invention relates to product selection using genetic analysis, and in particular to the selection of skincare, cosmetic, “cosmeceutical” and “nutricosmetic” products. The present invention also relates to genetic analysis to asses an indirect or direct-Response relationship between an active ingredient and its target to determine ingredient efficacy and more particularly, though not necessarily, to the case where such ingredients are ingredients within skincare, other cosmetic, “cosmeceutical” and “nutricosmetic” products.

### BACKGROUND

Many factors influence the health and appearance of skin tissue including genetics, diet, hormone levels, personal hygiene, and UV exposure from the sun. Experts have long recognised a list of “active” ingredients that play a key role in skin health. These critical skin enhancing ingredients include: a wide range of antioxidants, specific fatty acids other moisturizing agents, various vitamin, mineral co-factors and botanical elements including herbs and plant materials. The efficacy of an active ingredient relies on its ability to play a specific role in the biological pathway. This role is due to its capacity to interact with other molecules in the pathway to induce the desired response. Indeed an indirect or direct-Response relationship between an active ingredient and its target allows the ingredient to provide the best effect of a product.

Single-nucleotide polymorphisms (SNPs) are the most important and basic form of variation in the genome. They are responsible for individual differences in disease susceptibility and drug response. Detection of single-nucleotide polymorphism (SNP) to evaluate particular molecule functionality in a biological pathway is used in pharmaco-genetics in order to assess the whole body health status of a patient. SNPs are identified in a patient, taking into account the pathway of interest, in order to identify susceptibility to a disease. These methods are direct sourcing of a SNP, and can be seen as responding to the question: “Is this SNP associated with a disorder or a defect?”.

FIG. 1 shows a representation of a common process in which a molecule is tested for single-nucleotide polymorphism (SNP). This test is carried out to provide an outcome which will determine whether the person being tested has a susceptibility to a certain disease or not.

### SUMMARY

According to a first aspect of the present invention there is provided a method of assessing the suitability of a set of cosmetic and/or nutricosmetic and/or skin care products for an individual. The method comprises testing a sample of genetic material for an individual to identify the presence or absence of single-nucleotide polymorphisms at a predefined set of single-nucleotide locations. One or more weights for each location are identified in dependence upon the presence or absence of a single-nucleotide polymorphism at the location and the single-nucleotide location weights used in order to determine a product score for each of said products, a score being indicative of the suitability of a product to the individual.

The step of using the weights to determine a product score may involve combining the weights associated with those ingredients within a particular product.

The step of testing may comprise, in the event that a single-nucleotide polymorphism is present at a given single-nucleotide location, determining whether that single-nucleotide polymorphism is present in heterozygous or homozygous mutated form, and said step of identifying a weight or weights for each location comprises applying different weights to the heterozygous and mutated forms. For a given single-nucleotide location, the location is given a relatively high weighting if no single-nucleotide polymorphism is present, Wild Type, a relatively low weighting if a single-nucleotide polymorphism is present in homozygous mutated form, and an intermediate weighting if a single-nucleotide polymorphism is present in heterozygous form.

The weight or weights applied to a location may be dependent upon the level of interaction of an expressed gene, within which the single-nucleotide location is found, with an active ingredient. A location may be given a relatively low weight if a single-nucleotide polymorphism is present that is indicative of a function defect and may be given a relatively high weight if a single-nucleotide polymorphism is present that is indicative of a function gain.

The step of using the single-nucleotide location weights may comprise associating each of a predefined set of active product ingredients with one or more of said single-nucleotide locations, combining the location weights for the single-nucleotide locations associated with each active product ingredient to determine an ingredient score, and, for a given product, identifying the active ingredients in the product and determining said product score using the associated ingredient scores. The step of determining said product score may comprise identifying the number of active ingredients within a product that have a product score in excess of some predefined threshold score, and representing that number as a fraction or percentage of the total number of active ingredients within the product.

The step of identifying one or more weights for each location comprises determining different weights for different ingredients.

The method may comprise modifying the weights and/or scores in dependence upon lifestyle and/or extrinsic factors determined for the individual. It may comprise modifying the weights and/or scores in dependence upon ingredient dosage and or product composition.

According to a second aspect of the present invention there is provided a method of producing a cosmetic, nutricosmetic and/or skin care product tailored to an individual. The method comprises testing a sample of genetic material for an individual to identify the presence or absence of single-nucleotide polymorphisms at a predefined set of single-nucleotide locations, and identifying a weight or weights for each location in dependence upon the presence or absence of a single-nucleotide polymorphism at the location. The method further comprises associating each of a predefined set of active product ingredients with one or more of said single-nucleotide locations, combining location weights for the single-nucleotide locations associated with each active product ingredient to determine an ingredient score, selecting a subset of the active product ingredients using the ingredient scores, and mixing the subset of ingredients to produce a product for said individual.

According to a third aspect of the present invention there is provided a method of identifying one or more single-nucleotide polymorphisms, SNPs, that influence the efficacy of one or a combination of ingredients used in cosmetic, nutricosmetic and/or skin care products and which can be used to test for product suitability for users. The method comprises identifying one or a combination of genes associated with one or more biological pathways which in turn are influenced by the one or combination of ingredients, and for the or each gene, identifying SNPs that can be present within said gene(s). The method further comprises rating the identified SNPs to identify the SNP or SNPs that have a significant impact on the ability of the one or more biological pathways to be influenced by the ingredient(s).

The step of rating may take into account a number of properties and/or effects of the identified SNPs, one of the properties being the prevalence of a SNP within the user population or user population sub-group, wherein a SNP having a prevalence greater than some predefined threshold prevalence tends to be allocated a higher rating than a SNP having a prevalence lower than said threshold. Further properties that are taken into account during the step of rating may include one or more of the following: minor allele frequency, function population type, heterozygosis frequency and biological pathway.

The method may further comprise mapping information of the identified SNPs with a significant impact on the ability of the one or more biological pathways to be influenced by the ingredient(s), together with the ingredient(s) with which they are associated, and storing the mapped information in a database, such that it can be referred to during testing for product suitability for users.

The method may further comprise mapping the identified SNP(s) to cosmetic, nutricosmetic and/or skin care products that contain the ingredient(s) with which the identified SNP(s) is (are) associated, and storing the mapped information in a database, such that it can be referred to during product selection for users.

The one or combination of ingredients may be found to have reduced efficacy due to the presence of one or more identified SNPs within the or each gene associated with the or each biological pathways influenced by the one or combination of ingredients. The one or combination of ingredients may be found to have increased efficacy due to the presence of one or more identified SNPs because the one or more identified SNPs create a fault in one or more genes that is corrected by the ingredient.

According to a fourth aspect of the present invention there is provided a method of selecting a cosmetic, nutricosmetic or skin care product for a consumer and comprising: testing a biological sample obtained from the consumer to detect for SNPs identified using the method of the above third aspect of the invention, and selecting a cosmetic, nutricosmetic or skin care product from a range of available products on the basis of detected SNPs.

The product selection may also takes into account any synergistic effect of two or more ingredients working together. The step of testing the biological sample obtained from the consumer may comprise using primers selected to amplify the SNPs to be detected. The primers may be selected according to a number of criteria, the criteria including: primer length, the terminal nucleotide in the primer, reasonable GC (guanine-cytosine) content and Tₘ.

The methods presented above may be carried out using a DNA sequencing device coupled to one of a computer, portable computer device or mobile phone. The DNA sequencing device may be integrated into a USB stick. The method may be carried out using hardware in the form of a microchip, field programmable array or digital signal processor (DSP).

### BRIEF DESCRIPTION OF THE DRAWINGS

FIG. 1 is a representation of a process for testing a molecule for a SNP;

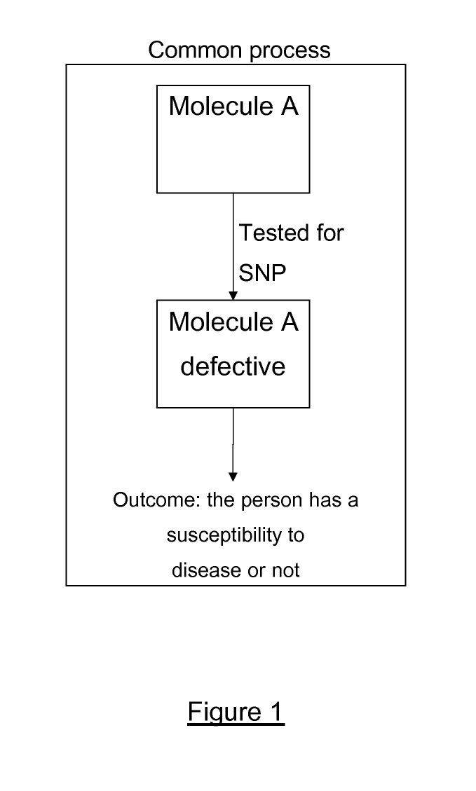

FIG. 2 is shows a signalling cascade illustrating activation of the biological pathway induced by an ingredient interacting with its biological target; and

FIG. 3 is a representation showing the selection of SNPs for ingredients working in synergy in the formulation of a skin care product, to provide advice to a customer;

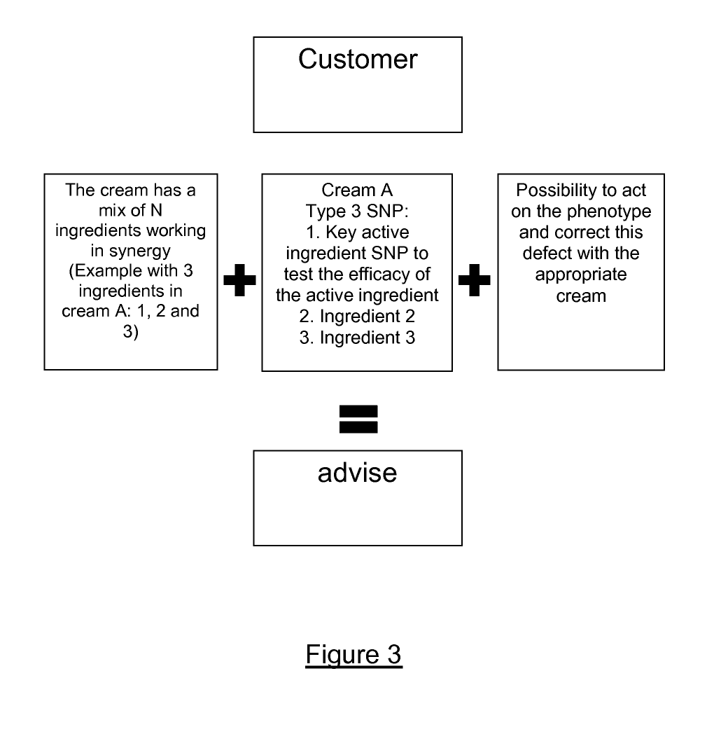

FIG. 4 is a flow chart illustrating a procedure for identifying SNPs that may be used to test for the efficacy of a particular ingredient; and

FIG. 5 is a flow diagram illustrating in general terms a method of determining product scores using a SNP detection procedure.

### DETAILED DESCRIPTION

In the following discussion, reference is made to “cosmetics” and cosmetic products. This terminology is intended to cover products such as skincare products and other products, such as “nutricosmetics” (i.e. nutritional supplements that are swallowed), that are intended to somehow interact with skin and other tissue to produce a beneficial effect that may be visible or invisible (e.g. improved comfort). The terminology also covers products that may not traditionally be referred to as cosmetics, for example, moisturising creams and aftershaves.

As previously discussed, experts have long recognised a list of “active” ingredients that play a key role in skin health. Of course new active ingredients are discovered from time to time. The genetic era now opens up the possibility to utilise a deeper understanding of genetics in a customised, i.e. person-specific, way. A single-nucleotide polymorphism (SNP) found in the target of an active ingredient provides information on the quality of the expected response, i.e. poor targeting equals poor response. Equally, an ingredient that is typically highly beneficial will have no effect if its target has a SNP associated with no functionality.

The predictive performance of an ingredient can be evaluated using cross-validation of its biological target. This method uses detection of SNP to estimate protein-protein interaction between an ingredient and its direct or indirect target. This approach is capable of finding dependencies between an ingredient's structural properties and its biological target and therefore an indication of the ingredient efficacy of a cosmetic product can be provided.

In order to use the detection of single-nucleotide polymorphism (SNP) as a tool to assess a cosmetic ingredient's efficacy to its specific target, a systematic method is required that associates an ingredient to a SNP in order to evaluate the efficacy of a selected biological pathway. As previously discussed, detecting SNPs to evaluate particular molecule efficacy in a biological pathway is known and is used in pharmacogenetics. However, the practice of identifying SNPs associated with an increased or decreased response to an active cosmetic ingredient (ACI) and then testing the individual to determine if they have at least one of said SNPs to determine whether that particularly cosmetic is likely to be effective for that individual, is new.

The method described herein is different from that used in pharmacogenetics in the sense that, instead of looking at whether a specific SNP is associated with a disorder or defect, the aim is to qualify the effect of an ingredient by querying the target of that ingredient, i.e. by identifying/assessing the presence or absence of SNPs in the targets associated with the active ingredient. By doing so, it is possible to determine if the ingredient will be efficient.

The method matches the ingredient to a SNP “strong” enough to affect the efficacy of the ingredient (i.e. the presence of the SNP has a considerable effect on the efficacy). The affect may be a reduction in efficacy, a total elimination of efficacy or increased efficacy. The direct target of ingredients (e.g. a receptor) within the biological pathway of interest is checked to see that it is functional. The functionality of the direct target is assessed by determining the presence or absence of a SNP that might distort the function of that target or enhance the function. A degree of impact, or “weight”, associated to a SNP is determined by a scoring method which will be explained in more detail below. These weights are typically not binary weights but rather have a degree of granularity.

There are six steps in the overall method:

Step 1: Identification of ingredients (within a range of cosmetic products) and their biological targets.
Step 2: Identification and selection of a SNP in the ingredient's target.
Step 3: Design of specific primers to amplify the specific SNP associated to ingredients.
Step 4: Matching ingredients to their target and the associated SNP.
Step 5: Correlation between ingredients and efficacy associated to SNP.
Step 6: Application: Selection of a group of SNPs associated with the composition of each cosmetic product (being considered) and its outcome.
These six steps will now be considered in more detail.
Step 1: Identification of Ingredients and their Targets

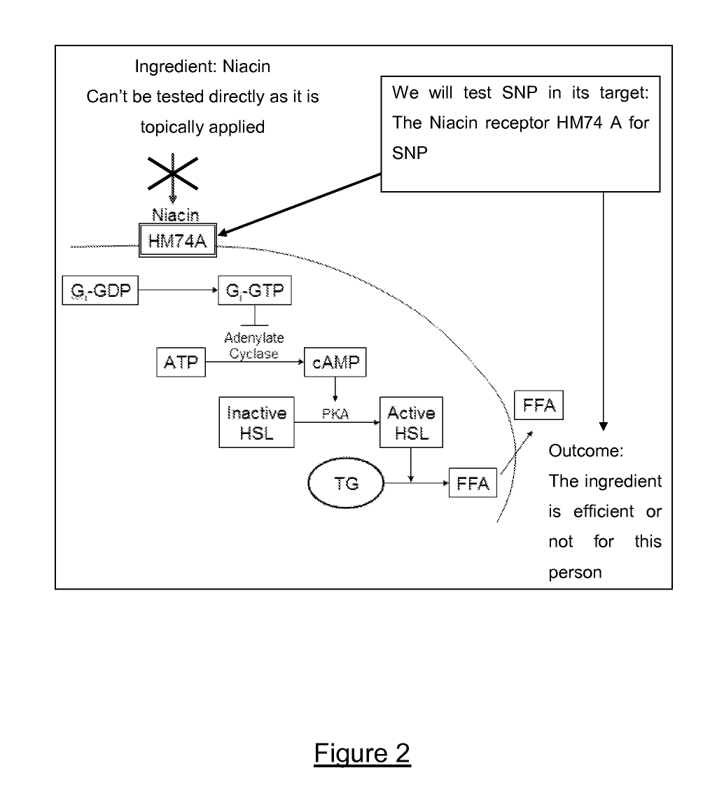

The skin's health is based upon 6 health categories or “pillars”, (sun screen, antioxidant, collagen stimulation, hydration and replenish). Each ingredient relates to one or more of these categories. Each ingredient is included in a product in order to take a specific biological pathway in the skin. These pathways are: antioxidant pathways for detoxifying, xenobiotic pathways, anti-ageing pathways and skin lightening pathways. Furthermore, each ingredient has one specific target (or biological target) in this pathway, which could be a direct and/or indirect target. A direct target is a molecule that has a physical interaction with the ingredient. The target is usually a protein with a key implication for the targeted biological pathway. The ingredient interacts with its biological target inducing a signalling cascade that is responsive of activation of this pathway, for example as shown in FIG. 2. The inclusion of genes in certain pathways can be based on information gathered from databases, for example the GeneCards® database and the KEGG GENES database, and also from selected publications. The genes have a proposed or established association with the listed ingredients and efficacy outcomes, but the genetic associations are not limited to the skin.

Take for example the normal aging process. Aging will eventually result in dermal and epidermal changes that will affect the structure and appearance of the skin. While many of the complex processes that affect the normal aging of the skin are still being researched, scientists now recognize oxidative damage as a major contributor to the aging process, and the single greatest cause of oxidative damage is UV radiation from exposure to sunlight. Scientists know that excessive exposure to sunlight can trigger inflammatory processes within the delicate skin tissues that result in an acceleration of the aging process. As the mechanism behind these oxidative processes has become better understood, we have now come to realize that the cumulative effects of even light to moderate exposure to UV radiation, over a lifetime, can cause significant pathological changes within the dermal and epidermal tissues. Since it is impossible to totally avoid exposure to solar radiation, it is important to get a greater understanding of these oxidative processes so that we can better protect our skin.

Skin tissues contain an amazing collection of enzymatic and nonenzymatic defence systems to help protect delicate dermal tissues from oxidative damage. These enzymatic systems include the following enzymes that are used as the target for cosmetic ingredients: Superoxide Dismutase, Catalase, Peroxidases, The Glutathione System, Thioredoxin Reductase, The Lipoamide System and NADPH Ubiqinone Reductase. The action mechanisms of these enzymatic systems are fairly well understood but are too complex to fully explain here. These systems are capable of protecting delicate tissues from the pathogenic effects of Reactive Oxygen Species and other Free Radicals associated with oxidative damage. These systems work by breaking down these free radicals before they have a chance to cause oxidative damage to the skin tissues specifically during skin ageing or while the skin is being damaged by the sun. However to be effective these molecules need a direct target. Therefore, the method comprises identifying a selection of SNPs that directly impact on these molecules by affecting their ability to respond to a specific cosmetic ingredient. In addition to the enzymatic systems, there are a number of non-enzymatic antioxidants, which also help to protect the skin. Well known non-enzymatic antioxidants include the following: Vitamin C, Vitamin E, Carotenoids including Beta Carotene and Lycopene, Bioflavonoids, Oligomeric Proanthocyanidins (e.g. Grape Seed Extract), Coenzyme Q10 and Polyphenols (e.g Green Tea). These antioxidants work synergistically with the enzymatic antioxidants to provide maximum protection from free radicals and the oxidative damage that they cause. Because each type of enzymatic and non-enzymatic antioxidant systems has its own unique features and effects, it is important that all of the systems are well represented and fully functional within the skin tissues. A list of typical cosmetic product ingredients and their biological effect is provided below.

### Step 2: Identification and Selection of SNP in the Ingredient's Target.

The ingredient's target corresponds to a protein related to a gene. Once the gene is identified, the SNP list for this gene is obtained, for example through NCBI website. The SNPs are then selected according to a list of key parameters indicating the relevance of specific SNPs. A list of main parameters to consider are:
Minor allele frequency: For a single-nucleotide polymorphism (SNP), its minor allele frequency (MAF) is the frequency of the SNP's less frequent allele in a given population. Minor allele frequency (MAF) refers to the frequency at which the less common allele occurs in a given population. SNPs with a minor allele frequency of 5% or greater were targeted by the HapMap project. MAF is widely employed in Genome Wide Association studies for complex traits.
Function: The consequence of the SNP at the protein level. Does it have an effect on the function of the protein?
Heterozygosis frequency: High heterozygosity means lots of genetic variability. Low heterozygosity means little genetic variability.
Major population: Population with the highest score for this SNP.
Biological pathway: Specification of the effect of the SNP on the biological system.
Gene ID: Gene corresponding to this SNP Link to NCBI

In order to determine the impact factor associated to a SNP, a method is carried out to assign a weight to each parameter by asking questions about the SNP. Each question regarding the key parameters for a SNP can be answered by a yes or a no. The aim of the question is to evaluate the occurrence of a key parameter for the SNP selection. If the answer is yes we assign one point, if the answer is no we assign zero points. Each of these parameters are equally important for the selection of a SNP. The end result is a score (1-5) representing the impact factor for each SNP, known as the SNP impact factor (SIF). The highest SIF score will indicate a high impact on the function (gain or loss) for the SNP used.

How the SIF values are evaluated:

1—The SNP has a poor effect on the ingredient.
2—The SNP has a weak effect on the ingredient.
3—The SNP will have mild effect on the ingredient.
4—The SNP has some moderate impact on the ingredient.
5 (Highest impact factor)—The SNP has a considerable effect on the ingredient.

For the matching process only SNPs with the score 5 are selected. Therefore, when SNPs are referred to herein, it typically means those with a SIF of 5, and they are sometimes referred to as functional SNPs. Examples of this can be seen in Table 1 below, which shows the SIF for MMP-1 associated with tocopherol and Collagen.

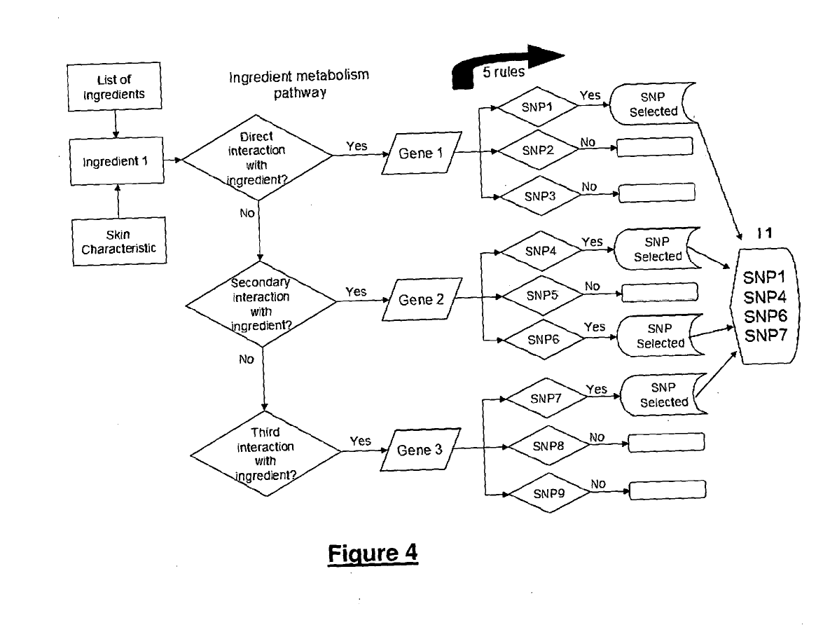

FIG. 4 is a flow chart illustrating a procedure for identifying SNPs that may be used to test for the efficacy of a particular ingredient (“ingredient 1”) within a particular product which contains a plurality of ingredients (the “List of Ingredients”). SNPs are determined for direct interaction of the ingredient metabolism pathway with the ingredient, as well as for secondary and third (tertiary) interactions. These levels can be set out as follows:
First level of interaction: First protein to interact directly with the ingredient (receptor or enzyme responsible for chemical interaction or ingredient modification). Severe consequence on the ingredient metabolism if this target is not functioning.
Second level of interaction: key molecule involved in the metabolism of the ingredients. moderate consequence on the ingredient metabolism if this target is not functioning.
Third level of interaction: Molecule involved in the metabolism of the ingredient but with minor consequence on the ingredient metabolism if this target is not functioning.

Secondary and tertiary levels of interaction could be interaction with, for example, an enzyme that functions within the metabolic pathway of the ingredient. For each level of interaction, the relevant gene and associated SNPs are identified. The five tests (questions) identified above are applied to determine whether or not a SNP should be selected. The outcome of the procedure is a set of SNPs, in this case {SNP1, SNP4, SNP6, SNP7}.

SNPs that are selected are typically well-represented within the whole population (e.g. greater than 5% frequency within the population). However, in some circumstances SNPs may be selected that are very specific to one or more groups within the population (i.e. smaller populations), and so may not actually be well-represented within the whole population.

Of course, SNPs may be selected that have either a beneficial influence on the metabolism of the ingredient (and therefore its efficacy), or conversely that may have a detrimental influence on the metabolism of the ingredient. Both of these situations are important to consider when selecting the SNPs.

### Step 3: Design of Specific Primers to Amplify the Specific SNP Associated to Ingredients.

One of the key aspects of this method is to design specific primers to target the right genotype. To amplify DNA, several standard methods can be used such as polymerase chain reaction (PCR), SNAP, or LAPM assay. All these techniques are based on the selection of accurate primers. Although the parameters used to select the right primers are known, the end results and the efficacy of the primer designed is new. The primers that have been designed for a few SNPs are described below. Primers were selected according to a number of criteria, including: primer length, the terminal nucleotide in the primer, reasonable GC content and Tₘ.

SNP genotyping methods have been developed such as polymerase chain reaction-restriction fragment length polymorphism (PCR-RFLP) analysis, the TaqMan PCR method, (Rychlik, et al, 1989) the Invader method (Lowe, T., J. et 1990), single-strand conformational polymorphisms analysis (Pallansch, L., et al, 1), allele-specific primer PCR analysis (Lucas, K., M. et al 1991) and allele-specific oligonucleotide hybridization analysis (Lucas, K., M. et al 1991). More recently DNA chip-based techniques are promising because they enable the simultaneous genotyping of many SNPs. Amplification of a specific DNA sequence is necessary for accurate SNP genotyping. DNA show high sequence homologies, it is essential to design the primers in the specific regions and to obtain the specific amplification product. Several nucleic acid amplification methods, including the PCR method or SNAP, or, have been developed so far (Dveksler, G. S., et al 1995; Ou, C.-Y. et al, 1988; Mack, D. H. et al, 1988). Among them, the loop-mediated isothermal amplification (LAMP) method (Dveksler, G. S., et al 1995) is very promising because the method can amplify DNA with high specificity and rapidity under isothermal conditions. Four specifically designed primers and a DNA polymerase with strand displacement activity are used for the amplification reaction.

The primer selection parameters described herein are general and are not necessarily implemented in the same manner among the different primer selection software. In addition, different programs attack the task of primer selection very differently, applying selection criteria to reduce the number of possible primers that the program must consider while not eliminating potentially good candidates. The unique combination of these parameters makes the primers unique.

Step 4: Matching Ingredients to their Target and the Associated SNP.

Each ingredient has the ability to be “metabolised” by a person. This ability is based upon the genetic makeup of this person. The metabolic pathways of many ingredients are identified and a list is created which details those ingredients that either become inactive due to the presence of a SNP, or a highly beneficial ingredient because the SNP creates a failing or fault that is corrected by this ingredient. The association between a SNP and the ingredient arises from the relationship between an ingredient and its biological target in the pathway. The ingredient influences the biological pathway, for example, the ingredient is metabolised in the pathway; or acts on elements of the pathway to thereby result in a phenotypic change. The use of a model of ingredient-target response efficacy has been found to be particularly beneficial.

Once the ingredients have been matched to their target and the associated SNP, this information can be entered into a table or database for future reference. This table can be extended in real time to reflect new ingredients that are discovered every day by the cosmetic industry. In order to increase the power of discrimination between products, the customer can be tested for more than one SNP to provide a full spectrum of efficacy within the product and the best combination of ingredients for their skin makeup. Tables 2 and 3 below show examples of the information gathered from the results of matching SNP to ingredients. Table 2 shows information for anti-aging ingredients, and Table 3 shows information for skin lightening ingredients.

Step 5: Correlation Between Ingredients and Efficacy Associated with SNPs.

It can now be determined if an ingredient is efficient or not when affected by a specific SNP(s) in its target. The final decision reflects the previously determined efficacy of the ingredient. If the target is not functional the ingredient will not be recommended. In contrast, if the target is not affected by the SNP the ingredient will be recommended. If the SNP provides again enhanced efficacy of an ingredient, the dosage of this ingredient might be considered before being recommended (especially if, at high dose, the ingredient is harmful, e.g. retinol). taken into consideration is the genotype identified by the test. This will affect the correlation given on the efficacy of the test.

### Step 6: Application: Selection of a Group of SNPs in the Composition of Cream and its Outcome.

The possibility of testing the SNPs associated to 3 main ingredients in a cosmetic product reflects the efficacy of the whole product. Some ingredients work in synergy, and so if one ingredient is not efficient due to a SNP in its target, the synergy expected between the ingredients will not occur. Therefore, it is desirable to test several of the product's ingredients according to the following method:

1. Assess the cream composition. Identify 3 or more ingredients in the mix.
2. Assessing the efficacy of the ingredient:
3. Test the efficacy of ingredients working in synergy in a skin care product.

The word synergy signifies that two ingredients or more are combined to produce an effect greater than the sum of their individual effects. In another words, the sum of the whole is greater than the individual parts, i.e. 1+1=3. This generally happens when the ingredients are complimentary to each other and together they give better results. The efficacy of each of these ingredients can be assessed by the method described above (i.e. Identifying associated SNPs) and identify each SIF. If one ingredient is not efficient (SIF=5) the synergy expected will not occur. Therefore the product will lose the synergy properties. For example, as vitamin C regenerates oxidized vitamin E, the combination in a cosmeceutical formulation is synergistic—particularly with regard to UV protection.⁵

FIG. 4 is a representation showing the selection of SNPs for ingredients working in synergy in the formulation of a skin care product, and the final advice given to the customer.

### Detailed Examples of the Matching Process, and how the SNP Result is Interpreted:

Interestingly a SNP will give different type of information, and a customised approach according to the consequence of each SNP must be adopted. A code is created for each outcome (e.g. recommended, not recommended, no added value etc.).

### Example 1

The cream Strivectin SD contains Niacin as an active ingredient. It is illustrated here how the related SNP is selected.

Possible Direct Target for Niacin as an Ingredient:

Information on the selection of possible targets for Niacin, in order to help the selection, can be found using established databases. For example, for Niacin this information can be found at http://www.t3db.org/toxins/T3D2841#target 1
1. Niacin receptor 1
2. Nicotinamide N-methyltransferase
3. Nicotinate-nucleotide pyrophosphorylase [carboxylating]
4. G-protein coupled receptor 109B

Once this information has been obtained, the question can be asked: “Niacin works through binding to its receptor. Is Niacin receptor-1 functioning?” An SNP is then looked for in the Niacin receptor-1.
Scientific evidence: HM74 (HUGO Gene Nomenclature Committee approved symbol: G protein-coupled receptor 109B [GPR109B]; MIM#606039), which codes for a putative Gi-G protein-coupled chemokine receptor, was recently identified as a receptor for niacin and was proposed as a mediator of n acin's effects on lipoprotein metabolism [Soga et al., 2003; Tunaru et al., 2003; Wise et al., 2003]. Population haplotype estimates derived from unselected control population suggest that HM74 and HM74A SNPs are not only frequent, but serve to discriminate the coding sequence of these genes. Previous studies suggest that these receptors are relatively unresponsive to niacin [Wise et al., 2003] in physiologic doses, and that pharmacologic doses are required to elicit response from these receptors. The impact of these SNPs (particularly the discriminating SNPs) on relative niacin response is therefore important, because discovery of potent and selective ligands for these receptors may be limited by differential pharmacologic responses based on the haplotype. The nonsynonymous nucleotide changes in HM74 (p.Phe198Leu, p.His253Arg; see FIG. 2) fall within two predicted transmembrane receptor (TMR) domains (TMR 5 and 6) and may lead to disorientation of these domains [Wise et al., 2003].
Selected SNP ID: HM74: p.His253Arg c.758A4G rs2454726c HM74: p.Phe198Leu c.592T4C rs7309297
SNP test outcome: If a mutation is found when the client is screened, it can therefore be determined that the cream Strivectin SD will be inactive for this person. The outcome is a loss of function.
Recommendation: This product will not be recommended (NR).

In order to increase the power of discrimination between products the customer can be tested with more than one SNP to provide a full spectrum of efficacy within the product and the best combination of ingredients for their skin makeup. The interface with the customer is flexible and it is possible to interrogate one type of skin care product according to the customer's need (for example, anti aging cream, Skin lightening cream, Collagen cream, etc.).

### Example 2

Customer A want to use a skin product Elemis® Pro-collagen Marine cream. It is known that this product contains Niacin in order to stimulate collagen production (this information is provided in a database). The customer can then be recommended one or more SNP tests to be carried out. In this example, the following SNP is suggested:
1. SNP1 MMP-1 (which is associated with collagen degradation)
The possible outcomes of the test are:
   Customer A has the mutated variant of SNP MMP1. The degradation of collagen is highly active in this person. In this case, all products with collagen are essential and highly recommended in addition to Elemis® Pro-collagen Marine cream. The customer could be provided with a list of other products if desired.
   Customer A has not got the mutated variant of SNP MMP1. The degradation of collagen is not an urgent issue for this person. In that case the product will be beneficial but not essential.

It will be appreciated that the genotype of an individual (including any SNPs of interest) will result in a certain phenotype when environmental factors are taken into account. In the present instance, such factors include the administration (or not) of an ingredient which, by acting on (or influencing) the pathway(s), results in a certain phenotype, or phenotypic outcome.

To help better understand the procedures involved in steps 4 to 6, a further example is now presented.

Each active ingredient is given a Weight according to the various genotypes, e.g. wild type (WT), mutated (Mut), and heterozygous (Het). Table 4 illustrates how the weightings are applied in the case of the active ingredient Niacin. Different weights are applied depending upon whether the level of interaction is a first, second or third level.

A higher weight represents a relatively more positive effect of a genotype whilst a lower weight represents a relatively less positive effect. In this example, Table 4 indicates that, for Niacin, for a given person, the presence of a WT genotype in any of the first to third levels of interaction suggests that Niacin will be very beneficial, whilst the presence of the mutated genotype for the first level of interaction will suggest that Niacin will be of little or no benefit. The presence of other genotypes (in the table) will indicate varying intermediate levels of benefit.

Weighting tables similar to Table 4 are constructed for all of the active ingredients in a product set of interest (e.g. for all skincare products made and sold by a given cosmetic company). These tables are integrated or made available, for example, into a point-of-sale terminal that is used by a sales person or beautician (“consultant”) that is assisting a customer to select a suitable product.

A sample of genetic material is obtained for an end user, e.g. customer, and that material tested to determine specific genotypes for the gene targets contained in the various weighting tables (e.g. Table 4). Table 5 illustrates the results, for a given individual, for two active ingredients, namely Niacin and Retinol. For each active ingredient, three gene types (SNPs) are tested for, but this number could be more or less depending upon the ingredient and the 5 step test (as per FIG. 4).

For each SNP, the maximum possible score is 10, so in this example (three SNPs) per ingredient, the maximum score for each ingredient is 30. It can be seen that, for this particular end user, ingredient 1 (Niacin) scores 15 out of 30, whilst ingredient 2 (Retinol) scores 13 out of 20.

The consultant could use these scores directly to advise a customer regarding selection of a suitable product. For example, the consultant might identify those ingredients that have the highest scores for the customer, and choose a product that contains those high scoring ingredients. The process might be implemented in a more automated fashion as follows.

For each product, only ingredients having a relatively high score (as per Table 5) are selected. Table 6 illustrates the scores obtained for three customers (A to C) and two products). The first product is a collagen boosting product, whilst the second is an anti-oxidant product. The collagen boosting product includes four active ingredients (1 to 4), whilst the anti-oxidant product also includes four active ingredients (5 to 8).

For the purpose of illustration, it is assumed that for each ingredient, three SNPs were selected, meaning that the maximum score for each ingredient is 30. [Of course, as already explained, more or less SNPs may be used for any given SNP giving rise to different maximum scores.] Using the results of Table 6, for each product those ingredients having a relatively high efficacy for an individual are selected. In this example, a score of 15 or greater indicates high efficacy, whilst a lower score indicates poor efficacy. This process is illustrated in Table 7 below, where the column titled “Ingredients Selected” identifies those ingredients, for each individual and each product, which have a relatively high efficacy. Using this data, a total product score is determined, for each person and each product, by determining the percentage of the total number of ingredients in a product that are considered to have a high efficacy. Thus, for the collagen boosting product for person A, ingredients 1 and 2 are considered to have a high efficacy, whilst ingredients 3 and 4 are considered to have a low efficacy, meaning that 50% of the ingredients have a high efficacy. This may be expressed as: POS=(sum ingredients selected/all ingredients)*100

It will be readily apparent from Table 7 that, for each person (customer), a consultant can rank products according to their relative benefits to that person. So, for example, for person A, the anti-oxidant product would be highly recommended whilst the collagen boosting product might not.

The SNP selection process considered in FIG. 4 may inherently account for genetic differences between populations, e.g. differences between Asian and European populations. Of course, additional account may be made for these differences by, for example, entering details of a customer's ethnicity into a point-of-sale terminal in order to tailor SNP selection or to adjust SNP weights.

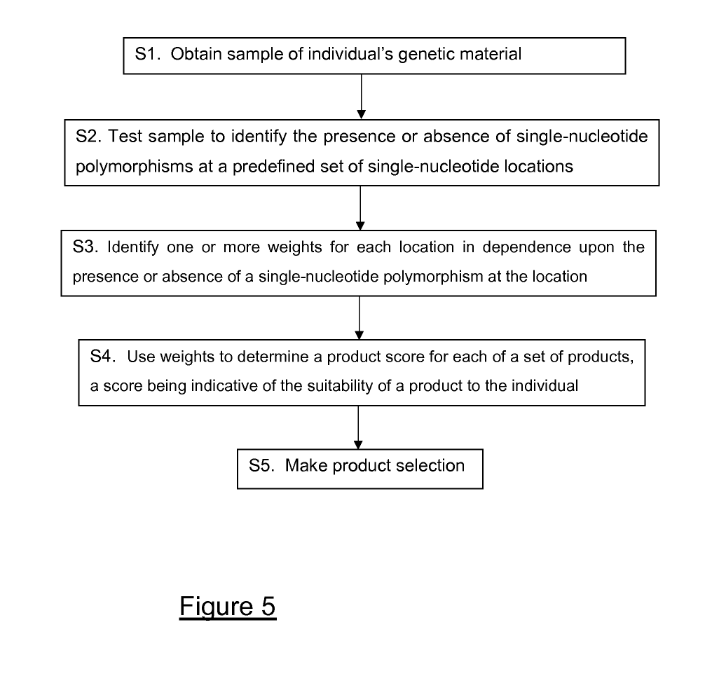

FIG. 5 shows a flow diagram illustrating in general terms a method of determining product scores, for each of a set of products, using a SNP detection process.

TABLE 1

| | | | The target | | | | High | |
|---|---|---|---|---|---|---|---|---|
| | | | is a Major | | Proven | Disruptive | Frequency | |
| | | | molecule | | Effect | effect | in the | Total |
| | | | in the | Direct | of the | for the | popula- | Score |
| Ingredient | Target | SNP ID | pathway | target | SNP. | ingredient | tion >0.05 | of yes |
| Niacin | HM74 | rs2454726 | yes | yes | yes | yes | yes | 5 |
| Collagen | MMP-11 | rs1799750 | yes | yes | yes | yes | yes | 5 |

TABLE 2

| Anti Aging | |
|---|---|
| Ingredients | SNP |
| NIA-114 ™ | Direct Target: Gene Niacin receptor |
| | SNP ID HM74: |
| | rs2454726c: p.His253Arg c.758A4G |
| | rs7309297: p.Phe198Leu c.592T4C |
| | Outcome: Mutant cannot metabolise Niacin |
| | Results: |
| | If mutant, the product is not efficient |
| | If not mutant product can be recommended |
| Pro-collagen | Direct Target: Collagen reparation |
| Padina Pavonica, | Indirect target: Gene MMP-1 |
| Triticum Vulgare | SNP ID: Rs1799750 |
| (Wheat) Germ Oil | Outcome: higher degradation of collagen |
| | Results: |
| | If the client is a mutant the product is very efficient |
| | and highly recommended |
| | If not mutant product is less recommended. |

TABLE 3

| Skin lightening | |
|---|---|
| Ingredients | SNP |
| Niacin | Direct Target: Niacin receptor gene |
| Niacin | SNP ID HM74: |
| | rs2454726c: p.His253Arg c.758A4G |
| | rs7309297: p.Phe198Leu c.592T4C |
| | Outcome: Mutant cannot metabolise Niacin |
| | Results: |
| | If mutant the product not efficient |
| | If not mutant the product can be recommended. |
| Quinone | Direct Target: NQO1 gene |
| | SNP ID: Rs1800566 |
| | Outcome: the NQO1 variant is in the active site of |
| | NQO1, leading to decreased NQO1 activity. NQO1 is a |
| | detoxification enzyme that catalyses the reduction of a |
| | range of substrates, particularly quinones. |
| | Results: |
| | If mutant, it is not possible to catalyse the quinone so the |
| | cream will not work. |
| | If not mutant - the cream will work. |

TABLE 4

| | | | | Consequence | |
|---|---|---|---|---|---|
| | Level of | Protein | Geno- | on ingredient | |
| Ingredient | Interaction | function | type | metabolism | Weight |
| Niacin | First | Full | WT | None | 10 |
| | (Niacin | Function | | | |
| | receptor) | Not | Mut | Severe | 0 |
| | | functioning | | | |
| | | Partially | Het | Moderate | 5 |
| | | functioning | | | |
| | Secondary | Full | WT | None | 10 |
| | | function | | | |
| | | Not | Mut | Substantial | 1 |
| | | functioning | | | |
| | | Partially | Het | Moderate | 6 |
| | | functioning | | | |
| | Third | Full | WT | None | 10 |
| | | Function | | | |
| | | Not | Mut | Significant | 2 |
| | | functioning | | | |
| | | Partially | Het | Moderate | 7 |
| | | functioning | | | |
| Vitamin D | First | Normal | WT | None | 10 |
| | (Vitamin D | Function | | | |
| | receptor) | Increased | Mut | Severe | 20 |
| | | function | | | |
| | | Partially | Het | Moderate | 15 |
| | | increased | | | |
| | | function | | | |
| | Secondary | Normal | WT | None | 10 |
| | | Function | | | |
| | | Increased | Mut | Substantial | 18 |
| | | function | | | |
| | | Partially | Het | Moderate | 14 |
| | | increased | | | |
| | | function | | | |
| | Third | Normal | WT | None | 10 |
| | | Function | | | |
| | | Increased | Mut | Significant | 16 |
| | | function | | | |
| | | Partially | Het | Moderate | 13 |
| | | increased | | | |
| | | function | | | |

TABLE 5
Personalised ingredients scoring. Ingredients will be
then ranked from the higher score to the smaller.

| | | | Level of | | |
|---|---|---|---|---|---|
| | | Genotype | inter- | | Total |
| Ingredient | Gene target | Person A | action | Weight | score |
| Ingredient 1 | SNP1 (rs1798192) | Mut | First | 0 | 15 |
| (Niacin) | SNP2 (rs17884481) | WT | First | 10 | |
| | SNP3 (rs17099455) | Het | First | 5 | |
| Ingredient 2 | SNP5 (rs1229966) | Mut | Second | 3 | 13 |
| (Retinol) | SNP6 (rs482284) | WT | Third | 10 | |

TABLE 6
Ingredients ranking and personalised according to the genotype.

| Characteristic | Ingredient (n) | Person A | Person B | Person C |
|---|---|---|---|---|
| Collagen Boosting | Ingredient1 | 15 | 30 | 21 |
| | (Niacin) | | | |
| | Ingredient 2 | 13 | 20 | 20 |
| | Ingredient 3 | 14 | 10 | 20 |
| | Ingredient 4 | 10 | 5 | 8 |
| Anti oxidants | Ingredient 5 | 12 | 26 | 14 |
| | Ingredient 6 | 24 | 25 | 28 |
| | Ingredient 7 | 21 | 26 | 27 |
| | Ingredient 8 | 18 | 23 | 12 |

TABLE 7

| | Ingredients | Ingredients | Total Product |
|---|---|---|---|
| Participant | properties | Selected | score |
| Person A- anti ageing test | Collagen | 2, 1 | 50% |
| | Boosting | | |
| | Anti oxidant | 6, 7, 8 | 75% |
| | Other see | | |
| | below | | |
| Person B- anti ageing test | Collagen | 1, 2 | 50% |
| | Boosting | | |
| | Anti oxidant | 5, 6, 7, 8 | 100% |
| Person C-anti ageing test | Collagen | 1, 2, 3 | 75% |
| | Boosting | | |
| | Anti oxidant | 6, 7 | 50% |

### REFERENCE

1. Wang X. A theory for the mechanism of action of the alpha hydroxy acids applied to the skin. Med Hypotheses 53(5):380-2 (1999 November).
2. Bickers D R, Athar M. Oxidative stress in the pathogenesis of skin disease. J Invest Dermatol 126:2565-75 (2006).
3. Beitner H. Randomized, placebo-controlled, double blind study on the clinical efficacy of a cream containing 5% alpha-lipoic acid related to photoageing of facial skin. Br J Dermatol 149(4):841-9 (2003 October)
4. Gaspar L R, Campos P M. Photostability and efficacy studies of topical formulations containing UV filters combination and vitamins A, C, and E. Int J Pharm 343(1-2):181-9 (2007 October).
5. Burke K E. Interaction of vitamins C and E as better cosmeceuticals. Dermatol Ther 20(5):314-21 (2007 September-October).
6. Bissett D L, Miyamoto K, Sun P, et al. Topical niacinamide reduces yellowing, wrinkling, red blotchiness, and hyperpigmented spots in aging facial skin. Int J Cosmet Sci 26(5):231-8 (2004 October).
7. Bissett D L, Oblong J E, Berge C A. Niacinamide: A B vitamin that improves aging facial skin appearance. Dermatol Surg 31(7 Pt 2):860-5 (2005 July).
8. Katayama K, Armendariz-Borunda J, Raghow R, et al. A pentapeptide from type I procollagen promotes extracellular matrix production. J Biol Chem 268(14):9941-4 (1993 May)
9. Robinson L R, Fitzgerald N C, Doughty D G, et al. Topical palmitoyl pentapeptide provides improvement in photoaged human facial skin. Int J Cosmet Sci 27(3):155-60 (2005 June).
10. Bissett D, Kimball A B, Robinson L, et al. Topical formulation containing N-acetyl glucosamine and niacinamide reduces the appearance of hyperpigmented spots on human facial skin. Presented at: the 64th Annual Meeting of the American Academy of Dermatology; San Francisco, Calif.; Mar. 3-7, 2006. Poster #P235.
11. Choi C M, Berson D S. Cosmeceuticals. Semin Cutan Med Surg 25(3):163-8 (2006 September).
12. Burke K E. Nutritional Antioxidants. In: Draelos Z D (ed). Cosmeceuticals. Philadelphia: Elsevier Saunders pp 125-32 (2005).
13. Hoppe U, Bergemann J, Diembeck W, et al. Coenzyme Q10, a cutaneous antioxidant and energizer. Biofactors 9(204):371-8 (1999).
14. Kim D W, Hwang I K, Kim D W, et al. Coenzyme Q10 effects on manganese superoxide dismutase and glutathione peroxidase in the hairless mouse skin induced by ultraviolet B irradiation. Biofactors 30(3):139-47 (2007).
15. Bauman L. Botanical ingredients in cosmeceuticals. J Drugs Dermatol 6(11):1084-8 (2007 November).
16. Saija A, Tomaino A, Trombetta D, et al. In vitro and in vivo evaluation of caffeic and ferulic acids as topical photoprotective agents. Int J Pharm 199:39-47 (2000 Apr. 10).
17. Tournas J A, Lin F H, Burch J A, et al. Ubiquinone, idebenone, and kinetin provide ineffective Photoprotection to the skin when compared to a topical antioxidant combination of vitamins C and E with ferulic acid. J Invest Dermatol 126:1185-7 (2006 May).
18. Murray J C, Burch J A, Streilein R D, et al. A topical antioxidant solution containing vitamins C and E stabilized by ferulic acid provides protection for human skin against damage caused by ultraviolet irradiation. J Am Acad Dermatol 59(3):481-25 (2008 September).
19. Khanna S, Venojarvi M, Roy S, et al. Dermal wound healing properties of redox-active grape seed proanthocianidins. Free Radic Biol Med 33(8):1089-96 (2002 Oct. 15).
20. Bagchi D, Bagchi M, Stohs S J, et al. Free radicals and grape seed proanthocyanidin extract: importance in human health and disease prevention. Toxicology 148(2-3):187-97 (2000 August).
21. Badreshia-Bansal S, Draelos Z D. Insight into skin lightening cosmeceuticals for women of color. J Drugs Dermatol 6(1):32-9 (2007 January).
22. Rendon M I, Gaviria J I. Skin lightening agents. In: Draelos Z D, ed. Cosmeceuticals. Philadelphia: Elsevier Saunders pp 103-9 (2005).
23. Dermal Safety. In: Gillner M, Moore G S, Cederberg H, et al. Environmental Health Criteria 157. Published under the joint sponsorship of the United Nations Environment Programme, the International Labour Organisation, and the World Health Organization. Available at: http://www.inchem.org/documents/ehc/ehc/ehc157.htm#SubSectionNumber:8.2 0.1. Last accessed Sep. 16, 2008.
24. Levitt J. The safety of hydroquinone: a dermatologist's response to the 2006 Federal Register. J Am Acad Dermatol 57(5):854-72 (2007 November).
25. Gupta A K, Gover M D, Nouri K, et al. The treatment of melasma: a review of clinical trials. J Am Acad Dermatol 55(6):1048-65 (2006 December)
26. Cosmetic Ingredient Review Expert Panel.Safety assessment of salixylic acid, butyloctyl salicylates, calcium salicylates, C12-15 Alkyl salicylates, capryloyl salicylic acid, hesyldodecyl salicylates, isocetyl salicylates, isodecyl salicylates, magnesium salicylates, MEA salicylates, ethylhexyl salicylates, potassium salicylates, methyl salicylate, sodium salicylates, TEA-salicylate, and tridecyl salicylate. Int J Toxicol 22(Suppl 3):1-108 (2003)
27. Buraczewska I, Berne B, Lindberg M, et al. Changes in skin barrier function following long-term treatment with moisturizers, a randomized controlled trial. Br J Dermatol 156(3):492-8 (2007 March).
28. Sorg O, Antille C, Kaya G, et al. Retinoids in cosmeceuticals. Dermatol Ther 19(5):289-96 (2006 September-October)
29. Oblong J E, Bissett D L. Retinoids. In: Draelos Z D, ed. Cosmeceuticals. Philadelphia: Elsevier Saunders pp 36-42 (2005).
30. Mukherjee S, Date A, Patravale V, et al. Retinoides in the treatment of skin aging: an overview of clinical efficacy and safety. Clin Intery Aging 1(4):327-48 (2006).
31. Pierard-Franchimont C, Castelli D, Van Cromphaut I V, et al. Tensile properties and contours of aging facial skin. A controlled double blind comparative study of the effects of retinol, melibiose-lactose and their association. Skin Res Technol 4:237-43 (1998).
32. Kafi R, Kwak H S, Schumacher W E, et al. Improvement of naturally aged skin with vitamin A (retinol). Arch Dermatol 143(5):606-12 (2007 May).
33. Creidi P, Vienne M P, Onchonisky S, et al. Profilometric evaluation of photodamage after topical retinaldehyde and retinoic acid treatment. J Am Acad Dermatol 39(6):960-5 (1998 December).
34. Rivers J K. Sunscreens. Skin Ther Lett Pharm Ed 2(1):6-7 (2007 March).
35. US FDA. FDA proposes new rule for sunscreen products. Available at: http://www.fda.gov/bbs/topics/NEWS/2007/NEW01687.html. Last accessed Sep. 15, 2008

1. A method of assessing the suitability of a set of cosmetic and/or nutricosmetic and/or skin care products for an individual, the method comprising:
testing a sample of genetic material for an individual to identify the presence or absence of single-nucleotide polymorphisms at a predefined set of single-nucleotide locations;
identifying one or more weights for each location in dependence upon the presence or absence of a single-nucleotide polymorphism at the location; and
using the single-nucleotide location weights in order to determine a product score for each of said products, a score being indicative of the suitability of a product to the individual.

2. The method according to claim 1, wherein said step of testing comprises, in the event that a single-nucleotide polymorphism is present at a given single-nucleotide location, determining whether that single-nucleotide polymorphism is present in heterozygous or homozygous mutated form, and said step of identifying a weight or weights for each location comprises applying different weights to the heterozygous and mutated forms.

3. The method according to claim 2, wherein, for a given single-nucleotide location, the location is given a relatively high weighting if no single-nucleotide polymorphism is present, Wild Type, a relatively low weighting if a single-nucleotide polymorphism is present in homozygous mutated form, and an intermediate weighting if a single-nucleotide polymorphism is present in heterozygous form.

4. The method according to claim 1, wherein the weight or weights applied to a location is or are dependent upon the level of interaction of an expressed gene, within which the single-nucleotide location is found, with an active ingredient.

5. The method according to claim 4, wherein a location is given a relatively low weight if a single-nucleotide polymorphism is present that is indicative of a function defect and is given a relatively high weight if a single-nucleotide polymorphism is present that is indicative of a function gain.

6. The method according to claim 1, wherein said step of using the single-nucleotide location weights comprises associating each of a predefined set of active product ingredients with one or more of said single-nucleotide locations, combining the location weights for the single-nucleotide locations associated with each active product ingredient to determine an ingredient score, and, for a given product, identifying the active ingredients in the product and determining said product score using the associated ingredient scores.

7. The method according to claim 6, wherein said step of determining said product score comprises identifying the number of active ingredients within a product that have a product score in excess of some predefined threshold score, and representing that number as a fraction or percentage of the total number of active ingredients within the product.

8. The method according to claim 1, wherein said step of identifying one or more weights for each location comprises determining different weights for different ingredients.

9. The method according to claim 1 and comprising modifying the weights and/or scores in dependence upon lifestyle and/or extrinsic factors determined for the individual.

10. The method according to claim 1 and comprising modifying the weights and/or scores in dependence upon ingredient dosage and or product composition.

11. A method of producing a cosmetic, nutricosmetic and/or skin care product tailored to an individual, the method comprising:
testing a sample of genetic material for an individual to identify the presence or absence of single-nucleotide polymorphisms at a predefined set of single-nucleotide locations;
identifying a weight or weights for each location in dependence upon the presence or absence of a single-nucleotide polymorphism at the location;
associating each of a predefined set of active product ingredients with one or more of said single-nucleotide locations, combining location weights for the single-nucleotide locations associated with each active product ingredient to determine an ingredient score;
selecting a subset of the active product ingredients using the ingredient scores; and
mixing the subset of ingredients to produce a product for said individual.

12. A method of identifying one or more single-nucleotide polymorphisms, SNPs, that influence the efficacy of one or a combination of ingredients used in cosmetic, nutricosmetic and/or skin care products and which can be used to test for product suitability for users, the method comprising:
identifying one or a combination of genes associated with one or more biological pathways which in turn are influenced by the one or combination of ingredients;
for the or each gene, identifying SNPs that can be present within said gene(s); and
rating the identified SNPs to identify the SNP or SNPs that have a significant impact on the ability of the one or more biological pathways to be influenced by the ingredient(s).

13. A The method according to claim 12, wherein said step of rating takes into account a number of properties and/or effects of the identified SNPs, one of the properties being the prevalence of a SNP within the user population or user population sub-group, wherein a SNP having a prevalence greater than some predefined threshold prevalence tends to be allocated a higher rating than a SNP having a prevalence lower than said threshold.

14. The method according claim 13, wherein further properties that are taken into account during the step of rating include one or more of the following: minor allele frequency, function population type, heterozygosis frequency and biological pathway.

15. The method according to claim 12, wherein the method further comprises mapping information of the identified SNPs with a significant impact on the ability of the one or more biological pathways to be influenced by the ingredient(s), together with the ingredient(s) with which they are associated, and storing the mapped information in a database, such that it can be referred to during testing for product suitability for users.

16. The method according to claim 12, wherein the method further comprises mapping the identified SNP(s) to cosmetic, nutricosmetic and/or skin care products that contain the ingredient(s) with which the identified SNP(s) is (are) associated, and storing the mapped information in a database, such that it can be referred to during product selection for users.

17. The method according to claim 12, wherein the one or combination of ingredients are found to have reduced efficacy due to the presence of one or more identified SNPs within the or each gene associated with the or each biological pathways influenced by the one or combination of ingredients.

18. The method according to claim 12, wherein the one or combination of ingredients are found to have increased efficacy due to the presence of one or more identified SNPs because the one or more identified SNPs create a fault in one or more genes that is corrected by the ingredient.

19. A method of selecting a cosmetic, nutricosmetic or skin care product for a consumer and comprising: testing a biological sample obtained from the consumer to detect for SNPs identified using the method of claim 1, and selecting a cosmetic, nutricosmetic or skin care product from a range of available products on the basis of detected SNPs.

20. The method as claimed in claim 19, wherein the product selection also takes into account any synergistic effect of two or more ingredients working together.

21. The method according to claim 19, wherein the step of testing the biological sample obtained from the consumer comprises using primers selected to amplify the SNPs to be detected.

22. A The method according to claim 21, wherein the primers are selected according to a number of criteria, the criteria including: primer length, the terminal nucleotide in the primer, reasonable GC (guanine-cytosine) content and Tₘ.
